(19) United States
(12) Patent Application Publication
(10) Pub. No.: US 2022/0386628 A1
(43) Pub. Date: Dec. 8, 2022

(54) CARBON DIOXIDE GENERATION

(71) Applicants: KATHOLIEKE UNIVERSITEIT LEUVEN, Leuven (BE); VIB VZW, Gent (BE)

(72) Inventors: Jan DELCOUR, Heverlee (BE); Lomme DELEU, Steendorp (BE); Kathleen HOOYBERGHS, Leuven (BE); Sarah PYCARELLE, Kortrijk (BE); Elias RAVIER, Kessel-Lo (BE); Joost SCHYMKOWITZ, Meensel-Kiezegem (BE); Frederic ROUSSEAU, Sint-Martens-Bodegem (BE); Rob VAN DER KANT, Heverlee (BE)

(21) Appl. No.: 17/775,031

(22) PCT Filed: Nov. 9, 2020
(86) PCT No.: PCT/EP2020/081479

§ 371 Date: May 6, 2022

(30) Foreign Application Priority Data
Nov. 7, 2019 (EP) 19207757.6

(51) Int. Cl. A21D 8/04; A21D 10/00; A21D 2/24; A21D 13/44; C12N 9/88

(52) U.S. Cl. CPC A21D 8/042; A21D 10/005; A21D 2/245; A21D 13/44; C12N 9/88; C12Y 401/01015

(57) ABSTRACT

The invention relates to methods of leavening a batter or dough of a bakery product comprising the steps of: providing a batter or dough comprising an isolated glutamate decarboxylase enzyme or an isolated aspartate decarboxylase enzyme and respectively glutamic acid or a salt thereof or aspartic acid or a salt thereof in a concentration of at least 0.005 mole glutamic acid/kg dough or batter, wherein the batter or dough does not contain added yeast or sourdough bacteria.

### FIELD OF THE INVENTION

The invention relates to the production of leavened food products such as cakes, cake doughnuts, muffins, cupcakes, pancakes, waffles and Irish soda breads.

The invention relates to the use of decarboxylating enzymes for the production of leavened food products.

### BACKGROUND OF THE INVENTION

Leavening agents provide many batter-based baked food products such as cakes, cake doughnuts, muffins, cupcakes, pancakes, waffles, but also dough based products such as Irish soda bread with proper airy structures.

The flour generally is supplemented with sodium bicarbonate (NaHCO₃) and (an) inorganic acid(s) [HX(s)]. These leavening agents release carbon dioxide (CO₂) as soon as they are in contact with each other. NaHCO₃ is very soluble in dough and batter aqueous phases. Different HXs are used to control CO₂ release, which itself in essence depends on when they dissolve. When CO₂ is released too early as a result of fast dissolving of HX, it diffuses through the dough or batter and is lost. When HX acts too late, products of low volume and poor structure are obtained. Very efficient HXs used in leavening systems are phosphate or aluminium based. However, their use is under increasing pressure because of health concerns.

Alternative methods for CO₂ generation in batter and dough are thus desired. Amino acid converting enzymes have been used in food applications for reducing acrylamide formation [asparaginase] and for the generation of gamma-aminobutyric acid (GABA) [glutamate decarboxylase]. Joye et al. (2011) Food Chem. 129, 395-401) describe the use of minute amounts of glutamic acid to increase GABA content in breakfast cereals. Lamberts et al. (2012) Food Chem. 130, 896-901) describe the use of glutamate decarboxylase to study GABA dynamics in bread making. Su. et al. (2011) Microb. Cell Fact. 10S1, S8) discuss glutamate decarboxylase activity of certain Lactobacillus strains used in sourdough bread making.

Recombinant glutamate decarboxylase is used for the industrial production of GABA. Mutants to change pH optimum and thermotolerance are known in the art.

### SUMMARY OF THE INVENTION

The invention relates to the production of different baked products (such as cakes, cake doughnuts, muffins, cupcakes, pancakes, waffles and Irish soda breads) with proper airy structures. Typically these products are obtained by the use of sweetened doughs.

This is done by using enzyme based leavening systems typically using an amino acid decarboxylase and its substrate. Doing so provides food products with a cleaner label, without metal based chemical leavening compounds, which at the same time can be enriched in compounds to which positive health effects have been ascribed, such as GABA or beta-alanine (BALA) in a bakery product. If such health effect or claim is not desired an enzyme can be used which generates L-alanine.

The present invention has the advantage that food acceptable enzyme substrates can be used (such as amino acids) and that food acceptable products can be obtained depending on the choice of enzyme and substrate.

The amount and timing of produced carbon dioxide can be adjusted by adapting the amount of substrate and/or enzyme, or by the choice of enzyme with respect to pH optimum and/or temperature optimum.

Chemical leavening is generally used as an alternative for yeast leavening to reduce the time needed to obtain a leavened product.

Whereas Lamberts et al. [cited above] disclose the use of glutamic acid and glutamate decarboxylase in bread dough and in a model system without added yeast, it could not be expected that such enzymatic process can generate sufficient amounts of carbon dioxide in the time period typically used for chemical leavening, and this in conditions which differ significantly from a buffered aqueous system.

The invention is further summarised in the following statements:
1. A method of leavening a batter or dough of a bakery product comprising the steps of:
   a) providing a batter or dough comprising an isolated food compatible decarboxylase enzyme and a substrate of said enzyme in a concentration of at least 0.003 mole substrate/kg dough or batter, and
   b) incubating said batter or dough under conditions allowing the generation of carbon dioxide by said enzyme.
2. The method according to statement 1, wherein step a) comprises adding substrate of said decarboxylase to the batter or dough and/or step a) comprises adding an isolated glutamate decarboxylase to the batter or dough.
3. The method according to statement 1 or 2, where the amount of substrate added to the dough or batter is between 0.004 and 0.20 mole/kg dough or batter.
4. The method according to any one of statements 1 to 3, wherein the bakery product is selected from the group consisting of an American biscuit, a cake, a cake doughnut, a cookie, a muffin, a pancake, a pretzel, a wafer, a waffle, and an Irish soda bread. Accordingly the methods typically use a sweetened dough to obtain such products.
5. The method according to any one of statements 1 to 4, wherein the dough or batter does not contain added yeast or does not contain added bacteria used in the production of sourdough.
6. The method according to any one of statements 1 to 5, wherein the dough or batter does not comprise reagents generating carbon dioxide via non-enzymatic reactions.
7. The method according to statement any one of statements 1 to 6, wherein the enzyme is an amino acid decarboxylase.
8. The method according to any one of statements 1 to 7, wherein the enzyme has a pH optimum between pH 3.0 and 8.0 or between 4 and 7.
9. The method according to any one of statements 1 to 8, wherein the enzyme has a temperature optimum between 20° and 90° C., between 20 and 30° C., between 30 and 40° C., or between 40 or 60° C.
10. The method according to any one of statements 1 to 9, wherein the enzyme is glutamate decarboxylase or aspartate decarboxylase or mixtures thereof.
11. The method according to any one of statements 1 to 10, wherein more than one glutamate decarboxylase is used, which differ from each other in pH optimum and/or in temperature optimum.
12. The method according to any one of statements 1 to 11, wherein the enzyme is glutamate decarboxylase.
13. The method according to any one of statements 1 to 12, wherein the enzyme is Streptomyces sp. glutamate decarboxylase, or Bacillus megaterium glutamate decarboxylase.
14. The method according to any one of statements 1 to 13, wherein the added substrate is glutamic acid and/or aspartic acid or a salt thereof [such as sodium glutamate and/or sodium aspartate].
15. The method according to any one of statements 1 to 14 where the substrate is generated by the chemical, physical or enzymatic conversion of a compound into the substrate.
16. The method according statement 15, wherein glutamic acid substrate is generated by the presence of glutathione or a salt thereof and glutathione hydrolase (EC 3.4.19.13).
17. Use of an isolated decarboxylase enzyme for leavening a dough or batter.
18. The use according to statement 17, wherein the decarboxylase enzyme is glutamate decarboxylase or aspartate decarboxylase.
19. A composition for the preparation of a bakery product comprising a cereal flour and/or an isolated starch, the composition having a water content below 15%, characterized by the presence of an isolated decarboxylase enzyme and a substrate of said enzyme wherein the concentration of said substrate is more than 0.005 mole substrate/kg composition.
20. The composition according to statement 19, wherein further comprising one or more of sugar, egg yolk, egg white, milk powder and cacao.
21. The composition according to statement 19 or 20, wherein the decarboxylase enzyme is glutamate decarboxylase or aspartate decarboxylase.
22. The composition according to, any one of statements 19 to 21, wherein the substrate is glutamic acid or a salt thereof, or aspartic acid or a salt thereof.
23. The composition according to any one of statements 19 to 22, which is a kit of ingredients wherein the enzyme and/or substrate are in separate packaging.
24. A dough or batter, characterized in the presence of an isolated food acceptable decarboxylase enzyme and a substrate of said enzyme at a concentration of at least 0.005, 0.010 or 0.20 mole substrate/kg dough or batter.
25. The dough or batter according to statement 24, wherein the decarboxylase enzyme is glutamate decarboxylase or aspartate decarboxylase.
26. The dough or batter according to statement 24 or 25, wherein the substrate is glutamic acid or a salt thereof, or aspartic acid or a salt thereof.
27. A method of leavening a batter or dough of a bakery product comprising the steps of:
   providing a batter or dough comprising an isolated glutamate decarboxylase enzyme or an isolated aspartate decarboxylase enzyme and respectively glutamic acid or a salt thereof or aspartic acid or a salt thereof in a concentration of at least 0.005 mole glutamic acid/kg dough or batter, wherein the batter or dough does not contain added yeast or sourdough bacteria.
28. The method according to statement 27, wherein step a) comprises:
adding glutamic acid or a salt thereof to the batter or dough comprising glutamate decarboxylase enzyme or
comprises adding aspartic acid or a salt thereof to the batter or dough aspartate decarboxylase enzyme.
29. The method according to statement 27 or 28, further comprising the heating of said dough or batter.
30. The method according to any one of statements 27 to 29 where the amount of a glutamic acid or a salt thereof a glutamic acid or a salt thereof or aspartic acid or a salt thereof in the dough or batter is between 0.010 and 0.20 mole/kg dough or batter.
31. The method according to any one of statements 27 to 30, wherein the bakery product is selected from the group consisting of an American biscuit, a cake, a cake doughnut, a cookie, a muffin, a pancake, a pretzel, a wafer, and a waffle.
32. The method according to any one of statements 27 to 31, wherein the dough or batter both comprises an isolated aspartate decarboxylase and an isolated glutamate decarboxylase and aspartic acid or a salt thereof and glutamic acid and a salt thereof.
33. The method according to any one of statements 27 to 31, wherein the dough or batter both comprises an isolated glutamate decarboxylase and does not comprise an isolated aspartate decarboxylase.
34. The method according to any one of statements 27 to 33, wherein more than one glutamate decarboxylase is used which differ from each other in pH optimum and/or in temperature optimum, and/or wherein more than one aspartate decarboxylase is used which differ from each other in pH optimum and/or in temperature optimum.
35. The method according to any one of statements 27 to 34, wherein the glutamate decarboxylase is Streptomyces sp. glutamate decarboxylase, or wherein the glutamate decarboxylase is Bacillus megaterium glutamate decarboxylase
36. Use of an isolated glutamate carboxylase and/or of an isolated asparate carboxylase in the leavening of a batter or dough of a bakery product, wherein said dough or batter does not contain added yeast or sourdough bacteria.
37. A dry mix comprising a cereal flour for the preparation of a bakery product, wherein the composition does not contain added yeast or sourdough bacteria, wherein the composition has a water content below 15% (w/w), characterized by the presence of an isolated glutamate decarboxylase enzyme and glutamic acid or a salt thereof in a concentration of more than 0.005 mole glutamic acid or its salt/kg composition and/or by the presence of an isolated aspartate decarboxylase enzyme and aspartic acid or a salt thereof in a concentration of more than 0.005 mole glutamic acid or its salt/kg composition.
38. The dry mix according to statement 37, further comprising one or more of sugar, dried egg yolk, dried egg white, milk powder and cacao.
39. The dry mix according to statement 37 or 38, comprising an a glutamate decarboxylase enzyme and glutamic acid or a salt thereof. and comprising an aspartate decarboxylase enzyme and aspartic acid or a salt thereof.
40. The dry mix according to any one of statements 37 to 38, which is a kit of ingredients wherein the enzyme and/or the amino acid substrate or its salt are in separate packaging.
41. A dough or batter which does not contain added yeast or sourdough bacteria, characterized by:
the presence of an isolated glutamate decarboxylase enzyme and glutamic acid or a salt thereof at a concentration of at least 0.005 mole glutamic acid or a salt thereof /kg dough or batter and/or
the presence of an isolated aspartate decarboxylase enzyme and aspartic acid or a salt thereof at a concentration of at least 0.005 mole aspartic acid or a salt thereof /kg dough or batter.
42. The dough or batter according to statement 41, comprising glutamic acid or a salt thereof at a concentration of at least 0.020 mole/kg dough or batter or comprising aspartic acid or a salt thereof at a concentration of at least 0.020 mole /kg dough or batter.

### DETAILED DESCRIPTION

### Figure Legends

FIG. 1: Height of pancake (PC) batters in function of time after being placed in a water bath at 50° C. for batter containing chemical leavening system (1.5 g NaHCO₃+1.5 g SAPP28); equimolar level of sodium glutamate (3.34 g) and 0.75 g NaHCO₃ [1Glu+0.5NaHCO₃ (pH 5.0)]; glutamate decarboxylase (GD), equimolar level of sodium glutamate (3.34 g) and 0.75 g NaHCO₃ [1Glu+GD+0.5 NaHCO₃ (pH 5.0)]; GD and equimolar dosage of sodium glutamate (3.34 g) [1Glu+GD (pH 5.0)]; GD and sodium glutamate in equimolar level X 2 (6.68 g) [2Glu+GD (pH 5.0)]; or GD and sodium glutamate in equimolar level X 2 (6.68 g) [2Glu+GD+sugar (pH 5.0)].
FIG. 2: Height of cream cake (CC) (batter) in function of time during baking in an electrical resistance oven (ERO) of cream cake containing no added leavening system (Negative control); chemical leavening system (NaHCO₃+SAPP28); sodium glutamate [equimolar amount X 2, 2Glu (pH 5.0)] or glutamate decarboxylase (GD) and sodium glutamate [equimolar amount×2, 2Glu+GD (pH 5.0)].
FIG. 3: Headspace CO₂ level as a function of time in an electrical resistance oven (ERO) during baking of cream cake (CC) batter containing no added leavening (Negative control); sodium glutamate [equimolar amount×2, 2Glu (pH 5.0)] or glutamate decarboxylase (GD) and sodium glutamate [equimolar amount×2, 2Glu+GD (pH 5.0)].
FIG. 4: Leavening of pancake batter GD ST: Streptococcus thermophilus glutamate decarboxylase; GD BM: Bacillus megaterium glutamate decarboxylase; Glu: sodium glutamate.
FIG. 5: leavening of cream cakes as function of baking time GD BM: Bacillus megaterium glutamate decarboxylase; Glu: sodium glutamate.
FIG. 6: headspace CO₂ level as a function of time in an ERO. GD BM: Bacillus megaterium glutamate decarboxylase; Glu: sodium glutamate.
FIG. 7: leavening of PC batter as a function of time PLP: Pyridoxal phosphate
FIG. 8: leavening of pancake batter with different pH values at 30, 50 and 70° C. as a function of time.

Food grade acids include citric, acetic, fumaric, lactic, phosphoric, malic or tartaric acid, sodium acid pyrophosphate (SAPP, Na₂H₂P₂O₇) monocalcium phosphate [MCP, Ca(H₂PO₄)₂], sodium aluminium phosphate (SALP) and sodium aluminium sulphate (SAS).

Flour is not a sterile product and may still contain traces of yeast and or bacteria. In the context of the present invention “without/no added yeast” and “without/no added “sourdough bacteria” refers to batters and dough containing flour which are not inoculated with additional bacteria or yeast.

The terms batter and dough are used as in Chapter 5 of Delcour JA and Hoseney RC, Principles of Cereal Science and Technology, AACC International, St. Paul, Minn., 2010. The products mentioned in the present paragraph are defined as in this reference. In the United States, cookies are products made from flour from soft wheat. They are characterized by a formula high in sugar and shortening and relatively low in water. Similar products made in Europe and the United Kingdom are called “biscuits.” The “biscuits” made in the United States (here referred to as American biscuits) are more accurately defined as chemically leavened bread. The diversity of cookie products is quite wide. They vary not only in formula but also in type of manufacture. Cookies can be classified according to the properties of their doughs. Hard doughs are related to bread dough since they have a developed gluten network, but they are of a stiff consistency. Short doughs, on the other hand, are much more like cake batters but contain much less water. Their consistency can be compared to that of wet sand, and, as a consequence, when pulled or under pressure, their structure breaks; i.e., it is short. These doughs have only limited, if any, gluten development. Perhaps the best way to classify cookies made from short doughs is by the way the dough is placed on the baking band. Such a classification allows us to divide cookies into three general types (rotary-mold cookies, cutting-machine cookies, and wire-cut cookies) such as also described in Delcour JA and Hoseney, RC [cited above]. The term ‘cake’ is used here for a whole gamut of food products which are described in Godefroidt et al. (2019) Comp. Rev. Food Sci. Food Safety 18, 1550-1562. Cake recipes typically list wheat flour, sugar, and eggs as ingredients. Depending on the cake type, lipids (e.g. margarine, oil, shortening, surfactants) are also part of the ingredient bill, as are ingredients such as leavening agent and salt. The term ‘cake’ is used for an extensive range of bakery products which differ strongly both in terms of their ingredients and ratios thereof, and in the processing methods used to manufacture them. There are different types of cakes. A first distinction is that between batter-type and foam-type cakes. Batter-type cakes (e.g. cream cake, pound cake) contain significant levels of fat. Their batters can be regarded as emulsions. Foam-type cakes, such as angel food and sponge cakes, contain only small levels of fat, as their recipes do not mention margarine, shortening, or oil. Their batters can be described as foams. However, the terminology used in literature is sometimes ambiguous, as the term sponge cake has been used for systems containing added fat. Additionally, cakes that can be considered to be a combination of foam-type and batter-type cakes, are sometimes referred to as chiffon cakes. A chiffon cake batter is both an emulsion and a foam. A second distinction is made between high-ratio and low-ratio cakes. While the recipes of the former contain more sugar than flour, those of the latter contain maximally as much sugar as flour. Angel food cake is an example of a high-ratio cake, while pound cake (or quatre quarts cake) is an example of a low-ratio cake. The term sponge cake has been used to describe both low-ratio and high-ratio cakes.

A cake doughnut is made from a sweetened dough that's leavened with the help of leavening agent. To obtain the product, the dough is cooked in oil into a product with a slightly crunchy exterior and a soft, cake-like interior.

A muffin is a small, round, sweet cake, usually with fruit or bran inside. It is often eaten for breakfast. In their production, muffin batter is often encouraged to overflow its baking cup, so that its top is larger in diameter, giving it somewhat of a mushroom shape.

A cupcake is a miniature cake. It is sweet, coming in flavours like vanilla, chocolate, and red velvet. It is tender and rich with eggs and butter.

A pancake (or hotcake, griddlecake, or flapjack) is a flat cake, often thin and round, prepared from a starch-based batter that may contain eggs, milk and butter and cooked on both sides on a hot surface such as a griddle or frying pan, often frying with oil or butter.

A waffle is a food product made from leavened batter or dough that is cooked between two plates that are patterned to give a characteristic size, shape, and surface.

Irish soda bread—or just soda bread—is a type of quick bread. In its traditional production, NaHCO₃ along with buttermilk are used as a leavening system. Such soda bread has four basic ingredients: flour, buttermilk, NaHCO₃ and salt. In less traditional ways of producing such bread buttermilk can (partly) be replaced by leavening acid.

The use of the word “bread” as such implies the presence of yeast.

Bread is typically prepared with 0 to 6 g sugar on 100 g flour. When such levels of sugar are used, part is converted by yeast into carbon dioxide and ethanol.

Doughs with sugar contents exceeding 10 g sugar on 100 g flour are classified as sweet doughs (Delcour and Hoseney 2010). “Sweet dough” accordingly encompasses doughs for the preparation of the above mentioned biscuits, American biscuits, cookies, and pretzels.

However, besides the use in the specific above mentioned Irish soda bread, the methods and compounds of the present composition are in principle equally applicable on recipes used in the preparation of bread (whole meal or sieved) of wheat, rye or other cereals or pseudocereals (such as buckwheat), whereby yeast is wholly or partially replaced by the enzymatic leavening system of the present invention. However, the application of the enzymatic leavening of the present invention is less preferred, or even discouraged. The taste and smell of a bread is in part obtained by metabolites produced by yeast. When the yeast is replaced by enzymatic leavening, this taste and smell is lost.

The aspect of smell and taste generated by yeast is less important or even not desired in, cakes, some pancakes and the like. Accordingly the enzyme based leavening system is particularly suitable for batters and sweet doughs.

The production of the above cakes, cake doughnut, muffin, cupcake, and pancake products to date rely on the action of chemical agents to ensure proper airy structures. This is also the case for some waffles and Irish soda breads. Commercial leavening agents generally contain NaHCO₃ and (an) inorganic acid(s) [HX(s)]. The salt and acid react with each other in the liquid phase of their batter/dough once they come into contact with each other. Depending on the type of leavening agent, the salt and HX used react during the batter/dough preparation (i.e. early-acting leavening agent) or during the early-baking phase (i.e. late-acting leavening agent). The most commonly used salt in cake making is NaHCO₃, due to its high solubility in aqueous media, its price, and its reactivity. Multiple HXs have been identified as of interest, based on the fact that their reaction with NaHCO₃ can be controlled. The main factor in deciding which acid to use is the desired moment of CO₂ release. Essentially, this depends on when the baking acid dissolves in the aqueous phase of the batter. NaHCO₃ reacts with acids according to the following reaction, in which a neutral salt (NaX), water (H₂O), and CO₂ are formed:

NaHCO₃+HX→NaX+H₂O+CO₂

When CO₂ is released prior to baking due to HX dissolving too fast, some if not all inflated gas cells diffuse through the batter and are lost at the surface. Significant gas cell coalescence can occur as well. Since CO₂ can only expand existing gas cells and not create new ones, the above can then result in e.g. cakes of low volume and/or coarse crumb.

In contrast, when HX acts too late, CO₂ is only released at the end of baking and thus at a moment when the gas cells are no longer able to expand because the cake matrix has already set at that point in time.

The HXs used are either inorganic or organic compounds. Inorganic HXs are preferred since they allow for better control of CO₂ release, while organic acids generally result in (too) early CO₂ release.

Single-acting leavening agents contain one HX (e.g. SAPP) and release CO₂ either earlier or later during the cake-making process. Double-acting leavening agents contain two HXs, typically an early and a late acting one, e.g. MCP and SALP, respectively.

However, CO₂ can also be formed due to thermal degradation of NaHCO₃ in the following reaction:

2NaHCO₃→Na₂CO₃+H₂O+CO₂

SALP has been one of the most commonly used inorganic baking HXs, due to it being heat-activated. It results in release of CO₂ mainly during baking and thus in high-volume food products. Whereas it is still a preferred HX, the use of aluminum in leavening agents is under pressure. In the EU, these slow-acting acids are listed on products label as E-numbers (e.g. E521 for SAS and E450 for SAP) along with that of NaHCO₃ (E500).

Several features of the current aluminium or phosphorous containing leavening acids contribute to the consumer-driven demand to no longer use them.

Some consumers detect a burnt (‘pyro’) taste in products from recipes containing them.

A significant group of consumers are on the outlook for food products that have a “clean label”, e.g. in Europe they avoid products with E-numbers on their label. The efficient higher temperature leavening agent SAS is increasingly under pressure because of health concerns. The same holds for phosphate based leavening acids. The above has already caused different retailers to forbid their use in food manufacturing.

It is an aim of the present invention to provide alternative leavening systems which do not rely on the use of above mentioned aluminium comprising compounds.

The present invention provides enzyme based technologies for generating CO₂ for leavening of inter alia cakes, waffles, pancakes, muffins and Irish soda bread. Typical embodiments use sodium glutamate decarboxylase (glutamate decarboxylase, EC 4.1.1.15) and/or sodium aspartate decarboxylase (aspartate decarboxylase, EC 4.1.1.11) enzymes in combination with their corresponding substrates.

Because enzymes are denatured during baked food production, they do not need to be listed on product labels as they are not considered to be additives.

Depending on the method of preparing the enzyme, it may be beneficial to add a cofactor to the dry dix or the batter or dough.

Therefore, the new technology is a cleaner label alternative for the use of current leavening systems containing at least two chemicals such as E500 and E521. Also, in some embodiments the technology provides for release of GABA and/or BALA. Many health effects have been described for these compounds. GABA lowers the human blood pressure and stimulates cancer cell apoptosis. BALA plays a role in skeletal muscle physiology. It has consistently been shown to improve high intensity exercise performance during high-intensity exercise bouts, attenuate neuromuscular fatigue in both men and women, and increase resistance training volume by enhancing the buffering capacity of skeletal muscle.

Alternatively, L-alanine is generated, to which no specific health effects are attributed.

The invention relates to the use of decarboxylase enzymes and their substrates for the creation of carbon dioxide in a dough or batter. In its broadest context decarboxylase enzyme relates to an enzyme of E.C. class 4.1.1. Whereas any enzyme and its substrate may be used for the generation of CO₂, it is understood by the skilled person that certain enzyme/substrate combinations are not suitable in the context of food preparation, in view of the smell, taste or health effect of traces of the substrate which may remain and of the product which is formed by the enzyme. Decarboxylase enzymes of which the substrate and the product formed are acceptable or allowed for food production are referred to as “food acceptable” enzymes.

More particularly decarboxylase enzyme relates to an enzyme which uses an amino acid as substrate.

Typically, enzymes for use in in the claimed invention are

Aspartate 1-decarboxylase (EC 4.1.1.11) converting L-aspartate into BALA+CO₂. Aspartate 4-decarboxylase (EC 4.1.1.12) converting L-aspartate into L-alanine+CO₂.

Glutamate decarboxylase (EC 4.1.1.15) converting L-glutamate in GABA+CO₂ In typical embodiments glutamate decarboxylase is E. coli glutamate decarboxylase A (Protein Accession Number: P69908) or E. coli glutamate decarboxylase B (Protein accession number P69910).

Glutamate decarboxylase is described as unusually specific, showing significant activity only on L-glutamic acid/glutamate and α-methyl glutamic acid/glutamate, whereas the following compounds are neither substrates nor inhibitors: D-glutamate, D- and L-aspartate, α-amino adipic and α-aminopimelic acids. Pure L-glutamine is not a substrate.

Glutamate decarboxylase exists as a hexamer of approximately 50 kDa identical subunits, each containing one molecule of pyridoxal phosphate (PLP), and has an optimal pH of 3.8.

Pyridoxal phosphate is a necessary but firmly bound coenzyme. Because of a reversible configurational change at about pH 5.5 (O'Leary & Brummund (1974) J. Biol. Chem. 249, 3737-3745), chloride ions may be stimulatory to activity and acetate ions may inhibit.

Inhibitors of E. coli GAD include L-isoglutamic acid, aliphatic dicarboxylic acids, especially glutaric, pimelic α-(fluoromethyl)glutamic acid and some sulfhydryl-group reagents such as mercuric chloride, pCMB, and DTNB.

In yet another specific embodiment the glutamate decarboxylase is from Streptococcus thermophilus. This enzyme has a temperature optimum of about 50°C.

In yet another specific embodiment the glutamate decarboxylase is from Bacillus megaterium.

In typical embodiments aspartate 1-decarboxylase is aspartate 1-decarboxylase of E. coli.

Aspartate 1-decarboxylase belongs to a class of enzymes that uses a covalently bound pyruvoyl prosthetic group.

Pyruvoyl-containing enzymes are expressed as a zymogen which is processed post-translationally by a self-maturation cleavage called serinolysis. E. coli contains two more such enzymes, phosphatidylserine decarboxylase and S-adenosylmethionine decarboxylase.

The PanD proenzyme (n protein) is processed at the serine residue at position 25, resulting in two subunits, α and β, which form a complex that is enzymatically active. Autocatalytic processing of purified enzyme preparations occurs slowly at room temperature or 37° C., and at a higher rate at elevated temperatures. An ester intermediate at Ser25, formed by an N—>O acyl shift, facilitates autoproteolysis β-elimination of the ester results in proteolysis and the formation of dehydroalanine, which undergoes hydrolysis to form the pyruvoyl group. Experiments in E. coli and Salmonella enterica have now shown that PanZ is a maturation factor that triggers cleavage of pro-PanD to its mature and active form.

For the purpose of the present invention, wherein isolated, recombinant enzymes are used, the enzymes may originate from fungi, plants or animals.

Enzymes are typically expressed in a bacterial expression system, not excluding expression in for example yeast, insect cells, or mammalian expression system. Enzymes may be intracellular expressed or secreted into the medium. Enzymes can be isolated from lysate or medium using chromatographic methods. Alternatively the enzyme may be expressed as a fusion protein with e.g. an His-Tag, as GST fusion, as MBP fusion. Depending on its effect on the activity of the enzyme, the tag may be removed from the enzyme using a protease cleavage site in the fusion construct. The selection of a particular enzyme is based on its compatibility with dough or batter and whether and how its catalytic activity is influenced by factors such as pH, temperature, sugar levels, ionic strength, and lipid contents.

The present invention envisages the use of different enzymes, converting different substrates, but both generating carbon dioxide (such as the use of glutamate decarboxylase and aspartate decarboxylase).

The present invention envisages the use of different variants of a same enzyme, whereby enzymes from different organisms are used (e.g. thermotolerant enzymes from microorganisms living in hot water) or wherein mutants of an enzyme are used which have been engineered for optimal performance under specific conditions of pH, temperature, sugar levels, ionic strength or lipid content of the dough or batter. Various engineered enzymes, known for use in other industrial applications, can be tested in the preparation of bakery products and compared with prior art chemically leavened products.

Amino acids which function as substrate can be provided in amino acid form or in a salt form (typically sodium salt) and/or hydrated form. Amino acids can be added as pure (>90% w/w, >95% w/w, 98% w/w) amino acid preparations, or alternatively as products comprising amino acids such as protein hydrolysates, or products such as quinoa flour or wheat bran preparations.

The activity of glutamate decarboxylase can be determined as in Joye et al. [cited above] by measuring the release of GABA from glutamic acid (or the loss of glutamic acid) as a function of time. This procedure can also be used for determining the activity of aspartate decarboxylase as the chromatographic conditions used by Joye et al. [cited above] are also suited to measure the enzymatically induced loss of aspartic acid as a function of time (Rombouts et al. (2009) J. Chrom. A 1216, 5557-5562). One enzyme unit of these enzymes is defined as the amount which converts 1.0 micromole of substrate per minute at 30° C. and pH 5.5. Enzyme units supplied are expressed per weight unit of the ingredient mixture, i.e. per weight unit of the sum of the weights of all solid and liquid recipe ingredients.

The same assay can be used to determine the activity of aspartic acid decarboxylase, by measuring consumption of aspartic acid and generation of BALA or L-alanine.

Chemical leavening requires the production of a certain volume of carbon dioxide, which differs depending on the type of envisaged bakery product. Lamberts et al. [cited above] discloses examples wherein the highest amount of sodium glutamate (Molecular Weight 169.1) is 380 ppm (i.e. 380 mg/kg flour), which corresponds to 2.25 mmole/per kg flour, or about 1.41 mmole per kg dough. The production of CO₂ under these conditions is too little to observe a significant increase in volume. Under complete conversion of the substrate and assuming no loss of CO₂ an increase in volume of only about 30 ml per kg dough would be produced. This would correspond to about 27 ml per litre dough as freshly mixed dough typically has a density of 1.1 kg/litre (Junge et al. (1981) Cereal Chem. 58, 338-342).

This is in strong contrast with the typical increase in volume of a yeast leavened bread where the volume increases by typically at least 2 litres per kg dough.

With an increase in volume by 10%, a litre of dough or batter contains 0.1 litre of CO₂ present as gas, which at room temperature equals to 0.00446 mole. Assuming a complete conversion of the substrate by the enzyme and no escape of CO₂ from the dough or batter, equally a minimum of 0.00446 mole (4.46 mmole) of substrate is required to obtain an increase in volume by 10% as some CO₂ is present in solution. With reference to the amino acids aspartic acid (Asp) or glutamic acid (Glu) as substrate an increase of 10% requires at least 600 mg Asp or 650 mg Glu per litre, as calculated based on the Molecular Weight of the amino acids. These weights further require adaptation when salts and/or hydrated forms of these amino acids are used.

Further assuming a complete conversion and no degradation during the baking process this results for glutamate decarboxylase in a release of 460 mg GABA and for aspartate decarboxylase 400 mg ala or BALA. Table 1 gives corresponding values when other increases in volume are considered.

TABLE 1
Substrates and products in enzymatic leavening as a function of
volume of CO₂ (in ml) generated per litre batter or 1.1 kg dough

| | | | | | g |
|---|---|---|---|---|---|
| | | | | | BALA |
| volume CO₂ | mmole CO₂ | gram Glu | gram GABA | gram Asp | or ala |
| 100 | 4.45 | 0.65 | 0.46 | 0.6 | 0.4 |
| 200 | 8.90 | 1.3 | 0.9 | 1.2 | 0.8 |
| 400 | 17.8 | 2.6 | 1.8 | 2.4 | 1.6 |
| 500 | 22.3 | 3.3 | 2.3 | 3 | 2 |
| 600 | 26.8 | 3.9 | 2.8 | 3.6 | 2.4 |
| 750 | 33.5 | 4.9 | 3.5 | 4.5 | 3 |
| 1000 | 44.6 | 6.6 | 4.6 | 5.9 | 4 |

Accordingly, the present invention relates to a batter or dough, and methods to prepare it, wherein a litre of batter or 1.1 kg of dough comprises at least 4, 6, 8, 10, 15, 20, 30, 40, 50, 75 or 100 mmole decarboxylase substrate.

Embodiments of the present invention relate to a batter or dough, and methods to prepare them, wherein a litre of batter or 1.1 kg dough comprises at least 0.5, 0.75, 1.0, 2, 4, 5, 7.5 or 10 g Asp or Glu (calculated as amino acid).

The present invention further relates to flour or to premixes of bakery products (i.e. flour further comprising one or more of sugar, dried egg yolk, dried egg white, sugar or cacao) comprising a substrate of a decarboxylase enzymes. These are dry compositions (i.e. comprising less than 15% v/w water) to which water or milk and other components are added. Accordingly the amount of substrate used for a litre of batter or a kg of dough can be present in a packaging as small as 100 g.

The invention accordingly relates to flour comprising at least 40, 60, 80, 100, 150, 200, 300, 400, 500, 750 or 1000 mmole decarboxylase substrate per kg flour.

The invention accordingly relates to flour comprising at least 5, 7.5, 10, 20, 40, 50, 75 or 100 g Asp or Glu (calculated as amino acid) per kg flour or premix.

The invention accordingly also relates to premixes comprising at least 20, 30, 40, 50, 75, 100, 150, 200, 250, 400 or 500 mmole decarboxylase substrate per kilogram premix.

The invention accordingly relates to flour comprising at least 2, 3, 4, 5, 10, 20, 25, 40 or 50 g Asp or Glu (calculated as amino acid) per kg premix.

The invention accordingly relates to bakery products comprising at least 4, 6, 8, 10, 15, 20, 30, 40, 50, 75 or 100 mmole of GABA per kg/bakery product.

The invention accordingly relates to bakery products comprising at least 4, 6, 8, 10, 15, 20, 30, 40, 50, 75 or 100 mmole of L-alanine or BALA per kg/bakery product. The invention accordingly relates to bakery products comprising at least 4, 6, 8, 10, 15, 20, 30, 40, 50, 75 or 100 mmole of L-alanine or BALA per kg/bakery product.

A flour or premix for use in the present invention can contain both decarboxylase enzyme and its substrate.

To prevent unwanted CO₂ formation by residual moisture, substrate and/or enzyme can be provided as separate packaging together with flour or premix and reconstituted.

In embodiments hereof, the enzyme is provided in a separate packaging and optionally comprises protein stabilizing agents such as albumin or starch or polysaccharides, comprises moisture absorbing compounds or comprises filling agents to avoid excessive loss of enzyme when packaged as a purified enzyme composition.

Different methods to formulate enzymes and products for use in flour are reviewed in EP 1224273 and include:
a) Spray dried products, wherein a liquid enzyme-containing solution is atomised in a spray drying tower to form small droplets which during their way down the drying tower dry form an enzyme-containing particulate material.
b) Layered products, wherein the enzyme is coated as a layer around a pre-formed inert core particle, wherein an enzyme-containing solution is atomised, typically in a fluid bed apparatus wherein the pre-formed core particles are fluidised, and the enzyme-containing solution adheres to the core particles and dries up to leave a layer of dry enzyme on the surface of the core particle.
c) Absorbed core particles, wherein rather than coating the enzyme as a layer around the core, the enzyme is absorbed onto and/or into the surface of the core.
d) Extrusion or pelletized products, wherein an enzyme-containing paste is pressed to pellets or under pressure is extruded through a small opening and cut into particles which are subsequently dried.
e) Products wherein an enzyme powder is suspended in molten wax and the suspension is sprayed, e.g. through a rotating disk atomiser, into a cooling chamber where the droplets quickly solidify.
f) Mixer granulation products, wherein an enzyme-containing liquid is added to a dry powder composition of conventional granulating components. The liquid and the powder in a suitable proportion are mixed and as the moisture of the liquid is absorbed in the dry powder, the components of the dry powder will start to adhere and agglomerate and particles will build up, forming granulates comprising the enzyme.

Since doughs and batters of the present invention typically do not contain yeasts, these can be provided as ready for use compositions wherein enzyme is to added to a liquid batter is mixed or stirred, or enzyme is added to dough and subsequently mixed or kneaded to disperse the enzyme in the dough.

### Example 1. Materials

Commercial white wheat flour (13.4% moisture content (MC) and 10% protein content), semi-skimmed milk, sugar, rapeseed oil and eggs were purchased at a local Belgian supermarket. The eggs were stored at 3° C. and used before their expiry date. L-Glutamic acid monosodium salt monohydrate (Molecular Weight 187,12) was from Fluka Honeywell (Morristown, N.J., USA). Citric acid, sodium chloride, silicon dioxide and L-aspartic acid sodium salt monohydrate (Molecular Weight 173,10) were from Merck (Darmstadt, Germany). NaHCO₃ and SAPP 28 (Sodium Acid Pyrophosphate) were from Budenheim (Budenheim, Germany). Sodium hydroxide was from J. T. Baker (Phillipsburg, N.J., USA). The cofactor pyridoxal 5′-phosphate hydrate (PLP or vitamin B6) was obtained from Merck (Darmstadt, Germany).

All chemicals were of at least analytical grade.

Aspartate decarboxylase was from Escherichia coli (Genbank Accession EFN38897.1). Glutamate decarboxylase was from Streptococcus thermophilus (Genbank Accession ABI31651.2) or from Bacillus megaterium (e.g. Genbank Accession KT895523.1) Both enzymes were expressed in E. coli as His Tagged proteins and purified by Ni-NTA affinity chromatography and supplied as solutions in 50 mM Tris-HCl buffer (pH 8.5) containing 300 mM sodium chloride.

### Example 2. In Vitro Release of Carbon Dioxide by Aspartate Decarboxylase and Glutamate Decarboxylase at Different pH Values

The CO₂ formation by glutamate decarboxylase and aspartate decarboxylase was measured at different pHs, both in the absence and presence of PLP. Citric acid buffers (50 mM) at pH 5.0, 6.0 and 7.0 contained 300 mM sodium chloride and 130 mM sodium glutamate or sodium aspartate. Test tubes containing 5.0 mL buffer and 0.1 g silicon dioxide (added as nucleating agent to readily detect CO₂ release) were incubated at 50° C. in a water bath for 10 min. Then, 100 μL glutamate decarboxylase or aspartate decarboxylase solution providing at least 13 enzyme units/g ingredient mixture and optionally a minute amount of cofactor PLP (between 2 and 5 mg) were added. Test tubes were vortexed to remove the bubbles initially present and put back in the water bath, which is the point in time at which observations started. For each pH, a negative control was performed in which no enzyme was used.

The following substrate/enzyme combinations were tested:

sodium aspartate/aspartate decarboxylase,

sodium glutamate/glutamate decarboxylase and

sodium aspartate/glutamate decarboxylase.

CO₂ formation was evaluated visually. Scores given were ‘0’ when no bubbles were noticed. A scale of ‘+’ to ‘++++’ was used to indicate little to intense bubble release, respectively.

In these experiments the test solutions did not include any of the components typically encountered in a batter or dough such as milk egg white or egg yolk, sugar or fat.

Table 2 provides information on the in vitro bubble formation by decarboxylation by glutamate decarboxylase and aspartate decarboxylase of their substrates during incubation at 50° C. in citric acid buffers (50 mM) at pH 5.0, 6.0 or 7.0 containing 300 mM sodium chloride and 130 mM sodium aspartate or sodium glutamate after addition of aspartate decarboxylase (AD) or glutamate decarboxylase (GD) and optionally cofactor pyridoxal 5′-phosphate (PLP) hydrate.

TABLE 2
Overview of bubble release. Scores were ‘0’ when no bubbles
were noticed. A scale of ‘+’ to ‘++++’ was used to indicate
little to intense bubble release, respectively.

| Enzyme | substrate | PLP | pH 5.0 | pH 6.0 | pH 7.0 |
|---|---|---|---|---|---|
| AD | Sodium aspartate | Yes | +++ | + | ++ |
| | | No | 0 | +++ | 0 |
| GD | Sodium glutamate | Yes | ++++ | + | ++ |
| | | No | ++++ | ++ | 0 |
| | Sodium aspartate | Yes | + | 0 | + |
| | | No | 0 | 0 | 0 |

The most intense gas release was observed for glutamate decarboxylase at pH 5.0 with sodium glutamate as substrate, irrespective of whether or not cofactor was added. Of all pH's tested, 5.0 is the closest to the reported optimum of this glutamate decarboxylase (pH 4.0). Glutamate decarboxylase was also active at pH 6.0 and 7.0 in this model system. Only little, but still significant gas release could be observed with sodium aspartate as substrate for glutamate decarboxylase.

Aspartate decarboxylase did induce gas release with sodium aspartate as substrate, but the overall gas release was less intensive than that obtained with glutamate decarboxylase with sodium glutamate. With aspartate decarboxylase, most gas release was also observed at pH 5.0. In this particular case, adding cofactor had an impact. Surprisingly, when no cofactor was added, gas release was most prominent at pH 6.0.

The above clearly showed gas release with the tested enzymes. Of the tested enzymes and conditions, glutamate decarboxylase in combination with sodium glutamate in a medium at pH 5.0 was most effective and therefore used for further experiments. While not necessary, PLP cofactor was added in all experiments in the subsequent examples.

### Example 3. Leavening of Pancake (PC) Batter by Glutamate Decarboxylase Activity

The following samples were prepared.

PC Batter 0:

175 g milk, 100 g flour, 100 g fresh egg white, 50 g fresh egg yolk and 1.5 g SAPP28 and 1.5 g NaHCO₃

All liquid ingredients were mixed in a Waring blender. After blending the liquids, the solids were mixed in. A graduated cylinder filled with 25 ml of the resulting batter was sealed with Parafilm and put in a water bath at 50° C. The increase in batter height was monitored over a 30 min period.

PC Batter 1:

159 g milk, 100 g flour, 100 g fresh egg white, 50 g fresh egg yolk, 6.68 g sodium glutamate, citric acid powder, glutamate decarboxylase solution (see below) and an aliquot of PLP (see below).

All liquid ingredients except for the glutamate decarboxylase solution were mixed in a Waring blender. The amount of milk was lower than in the reference batter to obtain a batter with the same liquid phase content as the reference batter once the glutamate decarboxylase solution had been added. After blending of all the liquids, flour was mixed into the formulation. Citric acid powder was added to obtain a batter at pH 5.0. A 50 ml graduated cylinder was filled with 10 ml of the resulting batter, a cofactor aliquot and 1.0 ml of a glutamate decarboxylase solution providing at least 4.2 enzyme units/g ingredient mixture were added. The cylinder was further filled with batter up to a volume of 25 ml, the cylinder contents were homogenized with a glass rod, the cylinder was sealed with Parafilm and put in a water bath at 50° C. The increase in batter height was monitored over a 30 min period.

The molar amount of sodium glutamate in Batter 1 is twice the molar amount of NaHCO₃ in pancake batter 0 (hereafter referred to as the Equimolar Amount×2).

PC Batter 2:

136.65 g milk, 31.8 g sugar, 100 g flour, 100 g fresh egg white, 50 g fresh egg yolk, 6.68 g sodium glutamate, citric acid powder, glutamate decarboxylase solution (as above) and an aliquot of PLP (as above). The amount of milk is reduced to compensate for the volume increase caused by the presence of sugar.

The preparation of batter 2 is the same as for batter 1, with the difference that both flour and sugar are added to the mixed liquids.

Batter 2 is prepared to determine whether sugar concentrations as encountered in food have an effect on glutamate decarboxylase activity.

PC Batter 3:

159 g milk, 100 g flour, 100 g fresh egg white, 50 g fresh egg yolk, 3.34 g sodium glutamate, citric acid powder, glutamate decarboxylase solution (as above) and an aliquot of PLP (as above).

Compared to batter 2, batter 3 contains half the amount sodium glutamate, in an molar equivalent of NaHCO₃ in the reference batter.

This concentration is hereafter referred to as the equimolar amount.

PC Batter 4:

159 g milk, 100 g flour, 100 g fresh egg white, 50 g fresh egg yolk and both 0.75 g

NaHCO₃ and 3.34 g sodium glutamate were used in the recipe.

This batter allows chemical leavening and enzymatic leavening.

PC Batters 5 to 9:

PC Batter 5 was made from the same recipe as batter 0, but without use of NaHCO₃ and without SAPP28.

PC Batter 6 was made from the same recipe as batter 1, but without use of glutamate decarboxylase and PLP.

PC Batter 7 was made from the same recipe as batter 2, but without glutamate decarboxylase and PLP.

PC Batter 8 was made from the same recipe as batter 3, but without glutamate decarboxylase and PLP.

PC Batter 9 was made from the same recipe as batter 4, but without glutamate decarboxylase and PLP.

FIG. 1 shows the leavening of PC batter as a function of time. Very efficient leavening was observed with both the equimolar amount×2 and the equimolar amount of sodium glutamate upon addition of glutamate decarboxylase. Sugar addition did not negatively impact the enzymatic leavening, it rather helped stabilizing the batter. A possible explanation is that sugar made the batter more viscous which then resulted in better retention of the formed CO₂.

In this model setting leavening was clearly faster acting than with SAPP28 as HX along with NaHCO₃. However, we performed this test at a temperature increasing from 23° C. (room temperature at the point of introduction in the water bath) to 50° C. (when at equilibrium with the water bath). SAPP28 may well be more active at higher temperatures only.

The combination of NaHCO₃ with glutamate decarboxylase and the lower dosage of sodium glutamate resulted in similar leavening than adding glutamate decarboxylase and equimolar×2 dosage of sodium glutamate. This volume increase was more than the sum of the leavening in batter with the equimolar dosage of sodium glutamate with glutamate decarboxylase and batter with NaHCO₃ (and sodium glutamate only). This model systems shows enzymatic leavening at higher temperatures. It also shows that glutamate decarboxylase can be used without significant inhibition (if any) by flour, egg white, egg yolk or milk components.

### Example 4: Leavening of Cream Cake (CC) Batter by Glutamate Decarboxylase Activity

The following samples were prepared:

CC Batter 0:

70.0 g flour, 59.5 g sugar, 42.0 g rapeseed oil, 31.5 g fresh egg white, 21.0 g fresh egg yolk, 28.0 g water and 1.05 g SAPP28 and 1.05 g NaHCO₃.

The solid ingredients were mixed in a Waring blender. Next, egg white, egg yolk, water, and rapeseed oil were blended in.

CC Batter 1:

70.0 g flour, 59.5 g sugar, 42.0 g rapeseed oil, 31.5 g fresh egg white, 21.0 g fresh egg yolk, 22.0 g water, 4.68 g sodium glutamate (i.e. equimolar amount×2), citric acid powder, 6.0 ml glutamate decarboxylase solution (see below) and between 2 and 5 mg of PLP.

The solid ingredients were mixed in a Waring blender. Next, egg white, egg yolk, water, and rapeseed oil were blended in. A small quantity of citric acid powder was added to obtain a batter at pH 5.0. Finally, 6.0 ml of a glutamate decarboxylase solution was mixed in which provided at least 2.5 enzyme units/g ingredient mixture.

CC Batter 2:

CC Batter 2 was made from the same recipe as batter 1, but without use of glutamate decarboxylase and PLP.

An electrical resistance oven (ERO; 75×60×180 mm, I×w×h) was filled with 150 g batter and sealed. A CO₂ data logger (CO₂Meter, Ormond Beach, Fla., USA) allowed monitoring CO₂-levels in the headspace. A ruler was used to monitor batter height during baking. The temperature-time profile was similar to that in the center during traditional cake baking. The temperature increased linearly from 25 to 90° C. in 23 min and then from 90° C. to 100° C. in 8 min and was finally held constant at 100° C. for 9 min.

FIG. 2 shows the leavening of cream cake as a function of baking time.

Glutamate decarboxylase in combination with its substrate did provide enzymatic leavening, which was in line with the results for pancake batter at 50° C. The leavening already was observable after 4 minutes of baking, i.e. at a temperature of 32° C. This was earlier than the tested chemical leavening which started after 8 minutes of baking (at about 46° C.). The results show that the technology can replace chemical leavening in bakery products.

FIG. 3 shows the headspace CO₂ level as a function of time in an ERO.

From 22 min of baking onwards a substantial amount of CO₂ was released from the cream cake batter when made with chemical leavening system or with glutamate decarboxylase and sodium glutamate. With only sodium glutamate, there was no production of CO₂ during the cake baking procedure, clearly showing the importance of added glutamate decarboxylase. The largest increases in CO₂ to the headspace were only seen at the beginning of the physical gas cell opening. Before this, the CO₂ remained captured in the bubbles of the batter, causing the leavening. The cell opening halted further increases in cake height and was related to the structure setting for regular leavening.

The results show that the leavening is effectively established by CO₂ production, be it enzymatically or chemically.

### Example 5. Leavening of Pancake (PC) Batter by Glutamate Decarboxylase Activity: Glutamate Decarboxylases from Streptococcus thermophilus and Bacillus megaterium

The following samples were prepared.

PC Batter 10:

159 g milk, 100 g wheat flour, 100 g fresh egg white, 50 g fresh egg yolk, 6.68 g sodium glutamate, citric acid powder (see below), solution of glutamate decarboxylase from Streptococcus thermophilus (ST, see below) and an aliquot of PLP (see below).

All liquid ingredients except the glutamate decarboxylase solution were mixed in a Waring blender. After blending, the solids were mixed into the formulation. Citric acid powder was added to adjust the batter pH to 5.0. A 50 ml graduated cylinder was filled with 10 ml of the resulting batter, an aliquot of PLP and 1000 μL of a solution of glutamate decarboxylase from ST providing a total of at least 4.2 enzyme units/g ingredient mixture were added. The cylinder was further filled with batter up to a volume of 25 ml, the cylinder contents were homogenized with a glass rod, the cylinder was sealed with Parafilm and put in a water bath at 50° C. The increase in batter height was monitored over a 30 min period.

PC Batter 11:

159 g milk, 100 g wheat flour, 100 g fresh egg white, 50 g fresh egg yolk, 6.68 g sodium glutamate, citric acid powder, solution of glutamate decarboxylase from ST and an aliquot of PLP (see below).

The preparation of batter 11 is the same as for batter 10 except that only 100.0 μL of the same glutamate decarboxylase solution was added which thus provided a total of at least 4.2 enzyme units/g ingredient mixture.

PC Batter 12:

159 g milk, 100 g wheat flour, 100 g fresh egg white, 50 g fresh egg yolk, 6.68 g sodium glutamate, citric acid powder, solution of glutamate decarboxylase from Bacillus megaterium (BM, see below) and an aliquot of PLP (see below).

The preparation of batter 12 is the same as for batter 10, with the difference that 100.0 μL of a solution of BM glutamate decarboxylase providing a total of at least 2.8 enzyme units/g ingredient mixture was added.

PC Batter 13:

PC batter 13 was made from the same recipe as batter 10, but without using glutamate decarboxylases. This batter is therefore a negative control for the other batters.

FIG. 4 shows the leavening of PC batter as a function of time.

Leavening was observed with the glutamate decarboxylase from ST as well as that from BM. When adding the BM glutamate decarboxylase extensive leavening was observed. The height of batter made with this enzyme increased to about 185% compared to 120% for batter made with the glutamate decarboxylase from ST. Also, the leavening of pancake batter when adding 1000 μl of glutamate decarboxylase solution from ST was less efficient than that of batter to which 100.0 μl glutamate decarboxylase solution from BM had been added.

Glutamate decarboxylase from BM caused pancake batter leavening already at room temperature as could be deduced from the height of the batter at 0 min. In the time frame between filling the cylinder with batter and putting it in the water bath at 50° C., batter height increased from 100% to about 108%.

These model systems show that enzymatic leavening at higher temperatures is possible with glutamate decarboxylases originating from different sources.

### Example 6. Leavening of Cream Cake (CC) Batter by Glutamate Decarboxylase Activity: Glutamate Decarboxylase from Bacillus megaterium

The following samples were prepared.

CC Batter 3:

70.0 g flour, 59.5 g sugar, 42.0 g rapeseed oil, 31.5 g fresh egg white, 21.0 g fresh egg yolk, 22.0 g water, 4.68 g sodium glutamate, citric acid powder.

The solid ingredients were mixed in a Waring blender. Next, egg white, egg yolk, water, and rapeseed oil were blended in. Citric acid powder was added to adjust the batter pH to 5.0.

CC Batter 4:

70.0 g flour, 59.5 g sugar, 42.0 g rapeseed oil, 31.5 g fresh egg white, 21.0 g fresh egg yolk, 22.0 g water, 4.68 g sodium glutamate, citric acid powder, 550 μL solution of glutamate decarboxylase from BM (see below) and an aliquot of PLP.

The solid ingredients except for the citric acid powder were mixed in a Waring blender. Next, egg white, egg yolk, water, and rapeseed oil were blended in. A small quantity of citric acid was added to adjust the batter pH to 5.0. After weighing 150 g batter in an electrical resistance oven, the cofactor PLP and the BM glutamate decarboxylase solution (which provided a total of at least 2.5 enzyme units/g ingredient mixture) were mixed in.

An electrical resistance oven (ERO; 75×60×180 mm, I×w×h) was filled with 150 g batter and sealed. A CO₂ data logger (CO₂Meter, Ormond Beach, Fla., USA) allowed monitoring CO₂ levels in the headspace. A ruler was used to monitor batter height during baking. The temperature-time profile was similar to that in the center during traditional cake baking. The temperature increased linearly from 25 to 90° C. in 23 min and then from 90° C. to 100° C. in 8 min and was finally held constant at 100° C. for 9 min.

FIG. 5 shows the leavening of cream cake as a function of baking time.

Glutamate decarboxylase solution from BM in combination with its substrate did provide enzymatic leavening during cream cake baking, which was in line with the results for pancake batter at 50° C. Batter height increased immediately after the start of baking and reached a maximum height of about 330% after 24 min of baking (at about 91° C.). This is much higher than leavening observed for cake batter made with the glutamate decarboxylase from ST where the batter reached a maximum height of about 260% (see example 4 t).

Due to the extensive leavening, the cake matrix was greatly stretched. As a result, cake structure collapsed when physical gas cell opening occurred. Hence, the used concentration of glutamate decarboxylase from BM was an overdose.

The results show that cream cake leavening can be reached by adding sodium glutamate in combination with glutamate decarboxylases originating from different sources.

FIG. 6 shows the headspace CO₂ level as a function of time in an ERO.

From 22 min of baking onwards a substantial amount of CO₂ was released from the cream cake batter when made with the BM glutamate decarboxylase and sodium glutamate.

At the end of baking much more CO₂ was released in the head space of the ERO when using glutamate decarboxylase from BM (ca. 57 000 ppm CO₂) rather than glutamate decarboxylase from ST (ca. 36 000 ppm CO₂, see example 4) for cream cake making further indicating the use of an overdose of the former.

With only sodium glutamate, there was no production of CO₂ during the cake baking procedure, clearly showing the importance of added glutamate decarboxylase. The results confirm that the use of sodium glutamate together with glutamate decarboxylase establish cream cake leavening during baking by CO₂ production.

### Example 7. Leavening of Pancake (PC) Batter by Glutamate Decarboxylase

Activity: Cofactor Dependency of Glutamate Decarboxylase from StreptoCoccus Thermophilus

Pancake batters were made with varying amounts of the cofactor PLP and equal amounts of glutamate decarboxylase solution from ST to examine whether glutamate decarboxylase activity depends on the amount of PLP present in the batter.

The following samples were prepared.

PC Batter 14:

159 g milk, 100 g flour, 100 g fresh egg white, 50 g fresh egg yolk, 6.68 g sodium glutamate (i.e. equimolar amount×2), citric acid powder and 250.0 μL solution of glutamate decarboxylase from ST (see below).

All liquid ingredients except the glutamate decarboxylase solution were mixed in a Waring blender. After blending of all liquids, the solids were mixed into the formulation. Citric acid was added to adjust the batter pH to 5.0. A 50 ml graduated cylinder was filled with 10 ml of the resulting batter after which a solution of glutamate decarboxylase from ST providing a total of at least 4.2 enzyme units/g ingredient mixture was added. The cylinder was further filled with batter up to a volume of 25 ml, the cylinder contents were homogenized with a glass rod, the cylinder was sealed with Parafilm and put in a water bath at 50° C. The increase in batter height was monitored over a 30 min period.

PC Batter 15:

PC batter 15 was made from the same recipe as batter 14, but with the addition of 5 mg PLP. PLP was added together with the glutamate decarboxylase solution.

PC Batter 16:

PC batter 16 was made from the same recipe as batter 14, but with the addition of 10 mg PLP. PLP was added together with the glutamate decarboxylase solution.

PC Batter 17:

PC batter 17 was made from the same recipe as batter 14, but with the addition of 25 mg PLP. PLP was added together with the glutamate decarboxylase solution.

PC Batter 18:

PC batter 18 was made from the same recipe as batter 14, but with the addition of 50 mg PLP. PLP was added together with the glutamate decarboxylase solution.

FIG. 7 shows the leavening of PC batter as a function of time.

Without adding PLP pancake batter leavening started after 5 min of incubation at 50° C., while with addition of PLP leavening started after 1 min of incubation. Cake batter to which the cofactor PLP and the glutamate decarboxylase solution were added, leavened faster than the batter to which only the latter was added.

The height of pancake batter made with PLP reached a maximum after about 10 min of incubation at 50° C. Thereafter, batter height remained constant for about 10 min and then slightly decreased at the end of the experiment. Pancake batter made without PLP reached its maximum height at the end of the experiment (i.e. 30 min) and would probably still have increased further when incubating for longer periods of time.

Pancake batter leavening was more outspoken with increasing PLP concentrations in the batter.

The results show that enzymatic leavening by the use of glutamate decarboxylase and its substrate in pancake batter depends on PLP concentrations in the batter.

### Example 8. Leavening of Pancake (PC) Batter by Glutamate Decarboxylase Activity: pH and Temperature Dependence of Glutamate Decarboxylase from Streptococcus thermophilus

The following samples were prepared.

PC Batter 19:

159 g milk, 100 g flour, 100 g fresh egg white, 50 g fresh egg yolk, 6.68 g sodium glutamate (i.e. equimolar amount×2), solution of glutamate decarboxylase from ST (see below) and an aliquot of PLP (see below).

All liquid ingredients except for the glutamate decarboxylase solution were mixed in a Waring blender. After blending all liquids, the solids were mixed into the formulation. Batter pH was 6.9.

12 ml of the batter was transferred to a Falcon tube after which 120.0 μl glutamate decarboxylase solution from ST and 24 mg PLP were added. After 1 min of vortexing 10 ml pancake batter was transferred to a 25 ml cylinder. The cylinder was sealed with Parafilm and put in a water bath at 30, 50 or 70° C. At every temperature, the increase in batter height was monitored over a 30 min period.

PC Batter 20:

PC batter20 was made from the same recipe as batter 19, but with addition of citric acid powder to adjust the batter pH to 6.0.

PC Batter 21:

PC batter 21 was made from the same recipe as batter 19, but with the addition of citric acid powder to adjust the batter pH to 5.0.

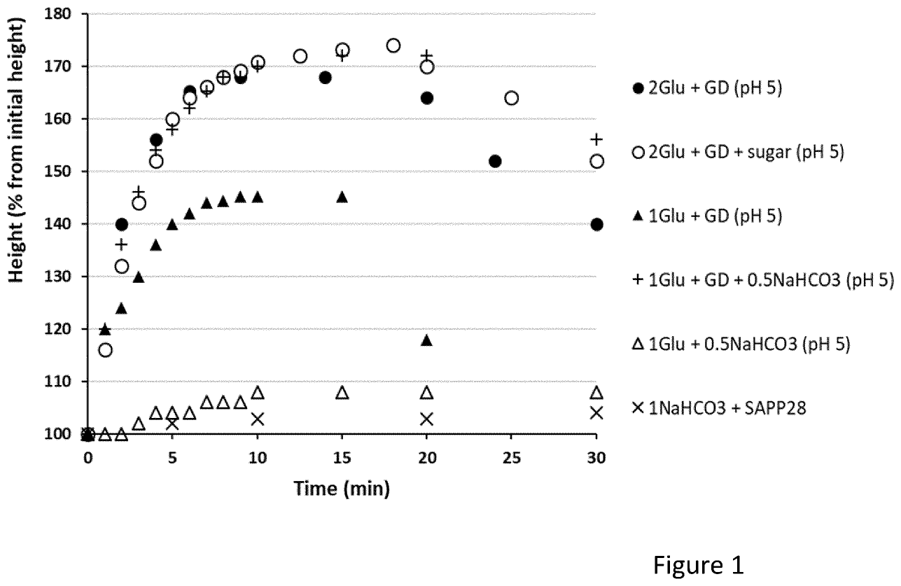

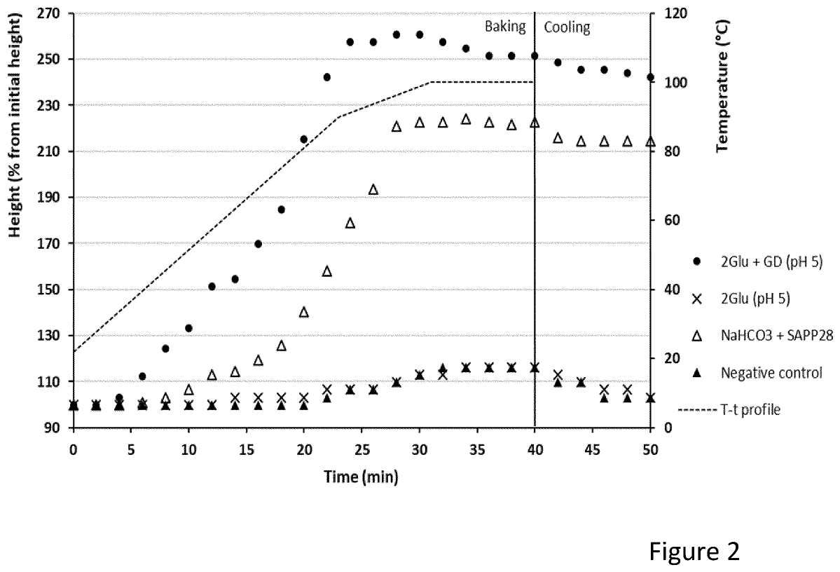

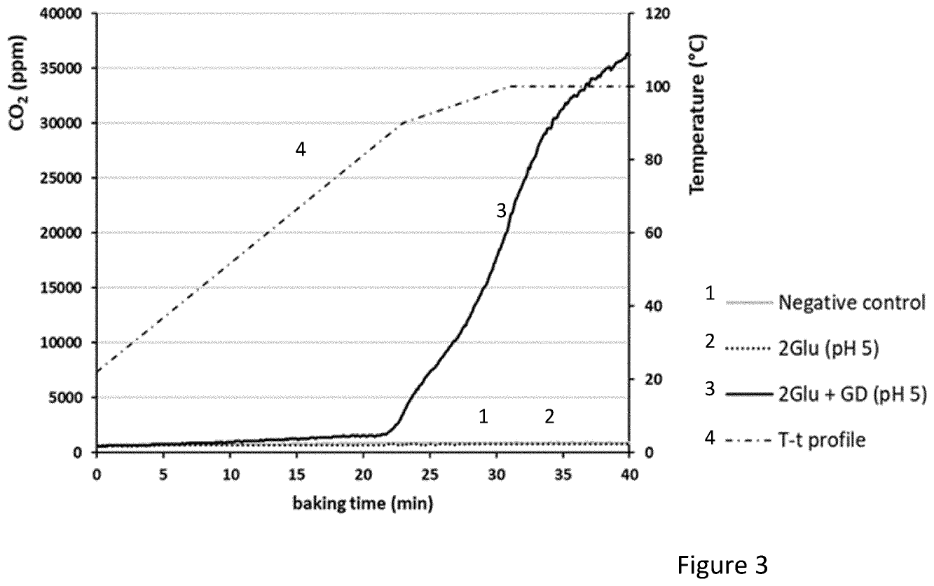

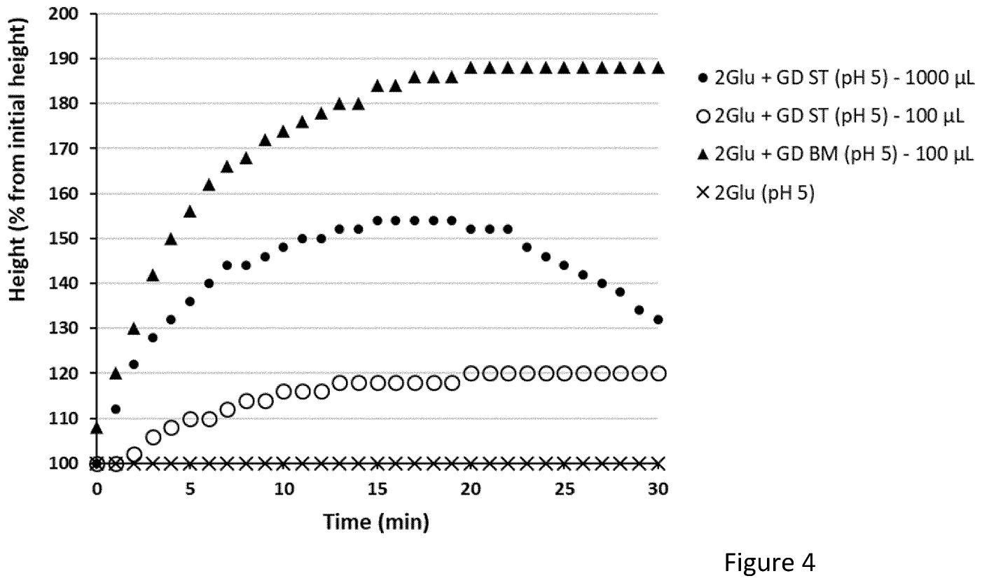

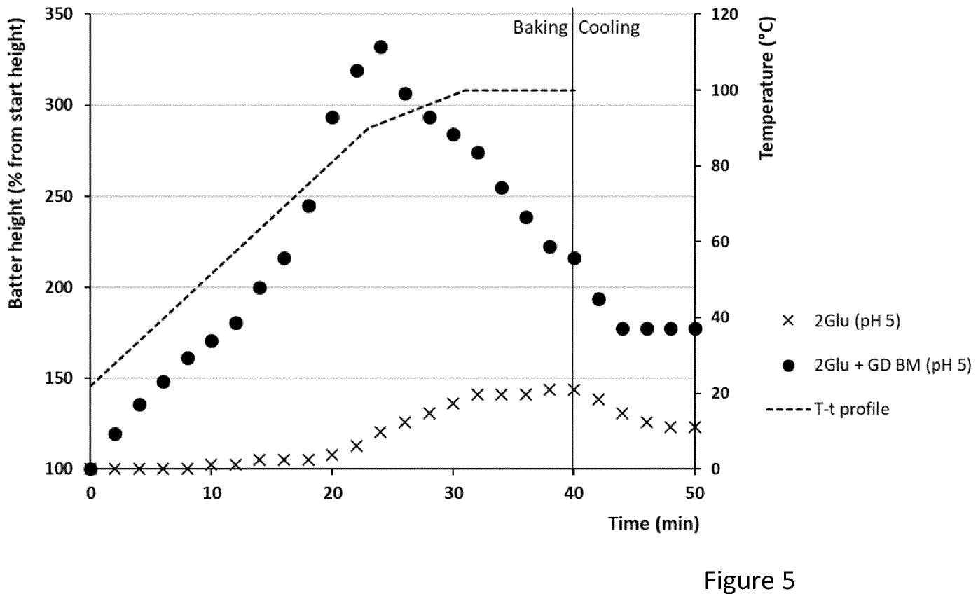

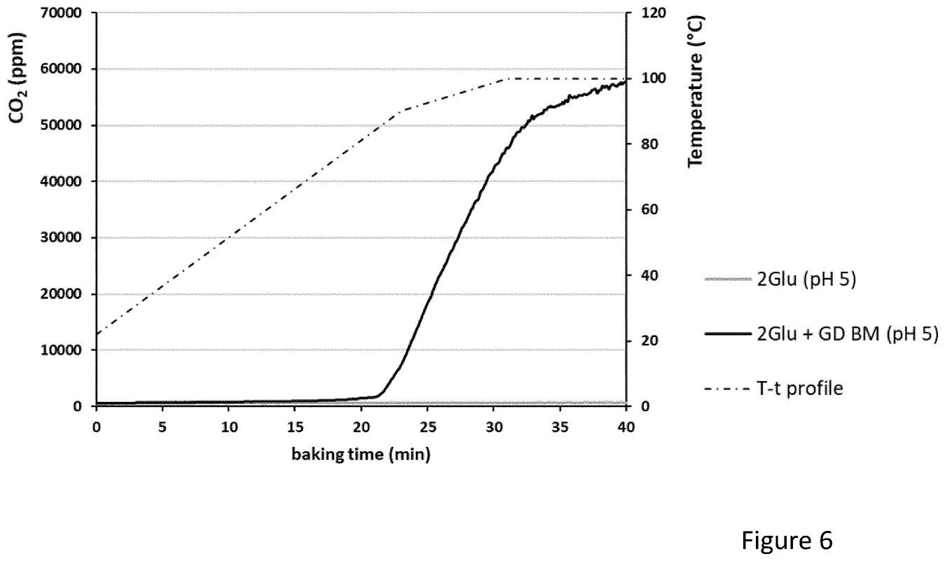

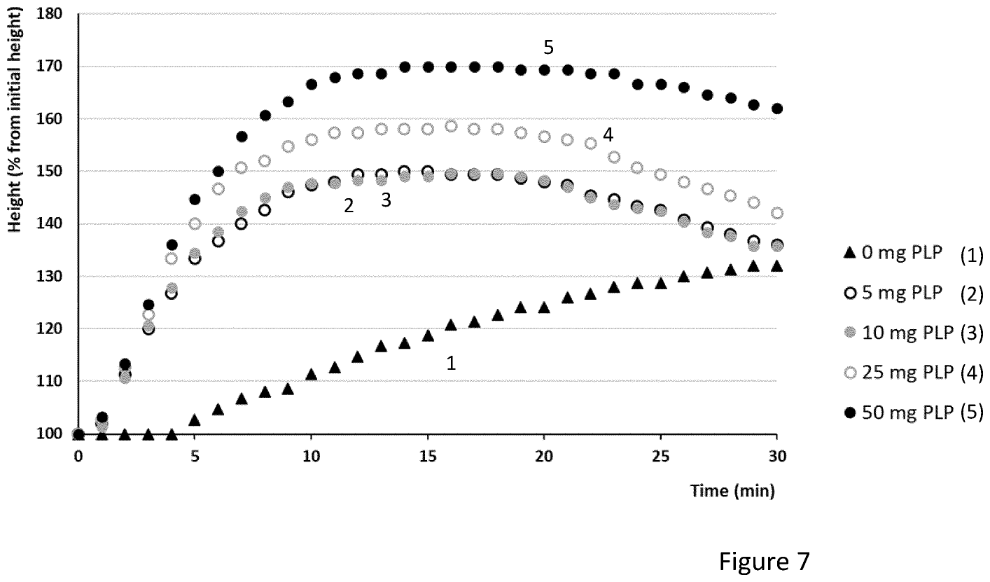

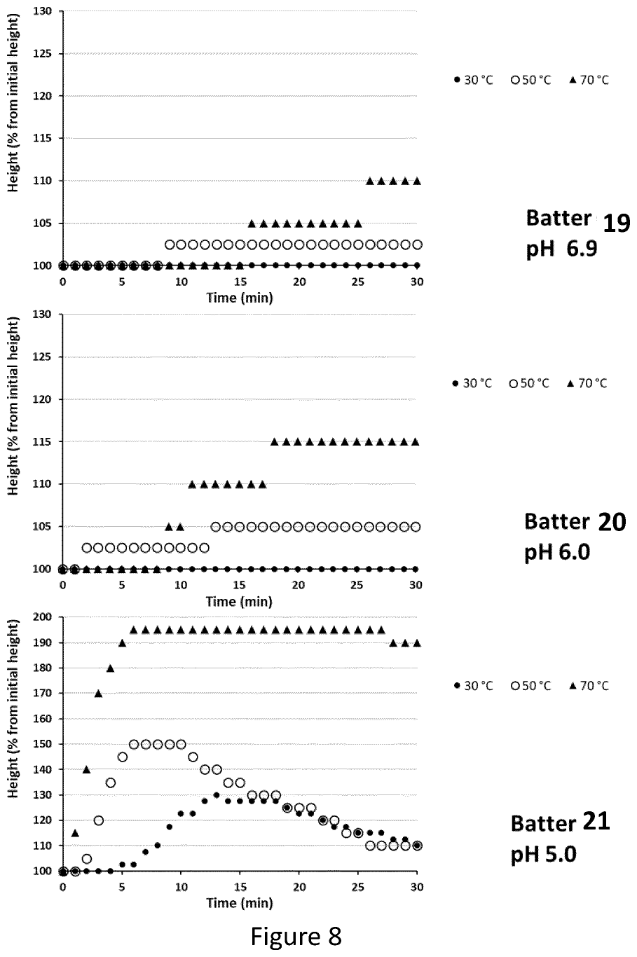

FIG. 8 shows the leavening of PC batter with different pH values at 30, 50 or 70° C. as a function of time.

Irrespective of batter pH, pancake batter leavening increased with increasing incubation temperatures. This is partly explained by a lower CO₂ solubility and thermal gas expansion at higher temperatures. In addition, glutamate decarboxylase activity likely increases with temperature. Its temperature optimum is about 50° C. (see page 10 original text).

Irrespective of the used incubation temperature, leavening of pancake batter with a pH of 6.9 or 6.0 was lower than that of pancake batter with a pH of 5.0. These results are partly explained by the fact that CO₂ can either occur as free CO₂ or as bicarbonate (HCO₃⁻). Their relative proportions depend on pH as described in Chapter 13 of Delcour JA and Hoseney RC, Principles of Cereal Science and Technology, AACC International, St. Paul, Minn., 2010. For instance, at pH 6 the relative proportions are approximately 70% CO₂ and 30% HCO₃⁻. Next to the above, glutamate decarboxylase activity likely also depends on batter pH. The results above are probably explained by a combination of both phenomena.

1. A method of leavening a batter or dough of a bakery product comprising the steps of:
providing a batter or dough comprising an isolated glutamate decarboxylase enzyme or an isolated aspartate decarboxylase enzyme and respectively glutamic acid or a salt thereof or aspartic acid or a salt thereof in a concentration of at least 0.005 mole glutamic acid/kg dough or batter, wherein the batter or dough does not contain added yeast or sourdough bacteria.

2. The method according to claim 1, wherein step a) comprises:
adding glutamic acid or a salt thereof to the batter or dough comprising glutamate decarboxylase enzyme or
comprises adding aspartic acid or a salt thereof to the batter or dough aspartate decarboxylase enzyme.

3. The method according to claim 1 or 2, further comprising the heating of said dough or batter.

4. The method according to any one of claims 1 to 3, where the amount of a glutamic acid or a salt thereof a glutamic acid or a salt thereof or aspartic acid or a salt thereof in the dough or batter is between 0.010 and 0.20 mole/kg dough or batter.

5. The method according to any one of claims 1 to 4, wherein the bakery product is selected from the group consisting of an American biscuit, a cake, a cake doughnut, a cookie, a muffin, a pancake, a pretzel, a wafer, and a waffle.

6. The method according to any one of claims 1 to 5, wherein the dough or batter both comprises an isolated aspartate decarboxylase and an isolated glutamate decarboxylase and aspartic acid or a salt thereof and glutamic acid and a salt thereof.

7. The method according to any one of claims 1 to 5, wherein the dough or batter both comprises an isolated glutamate decarboxylase and does not comprise an isolated aspartate decarboxylase.

8. The method according to any one of claims 1 to 8, wherein more than one glutamate decarboxylase is used which differ from each other in pH optimum and/or in temperature optimum, and/or wherein more than one aspartate decarboxylase is used which differ from each other in pH optimum and/or in temperature optimum.

9. The method according to any one of claims 1 to 8, wherein the glutamate decarboxylase is Streptomyces sp. glutamate decarboxylase, or wherein the glutamate decarboxylase is Bacillus megaterium glutamate decarboxylase

10. Use of an isolated glutamate carboxylase and/or of an isolated asparate carboxylase in the leavening of a batter or dough of a bakery product, wherein said dough or batter does not contain added yeast or sourdough bacteria.

11. A dry mix comprising a cereal flour for the preparation of a bakery product, wherein the composition does not contain added yeast or sourdough bacteria, wherein the composition has a water content below 15% (w/w), characterized by the presence of an isolated glutamate decarboxylase enzyme and glutamic acid or a salt thereof in a concentration of more than 0.005 mole glutamic acid or its salt/kg composition and/or by the presence of an isolated aspartate decarboxylase enzyme and aspartic acid or a salt thereof in a concentration of more than 0.005 mole glutamic acid or its salt/kg composition.

12. The dry mix according to claim 11, further comprising one or more of sugar, dried egg yolk, dried egg white, milk powder and cacao.

13. The dry mix according to claim 11 or 12, comprising an a glutamate decarboxylase enzyme and glutamic acid or a salt thereof.
and comprising an aspartate decarboxylase enzyme and aspartic acid or a salt thereof.

14. The dry mix according to any one of claims 11 to 13, which is a kit of ingredients wherein the enzyme and/or the amino acid substrate or its salt are in separate packaging.

15. A dough or batter which does not contain added yeast or sourdough bacteria, characterized by:
the presence of an isolated glutamate decarboxylase enzyme and glutamic acid or a salt thereof at a concentration of at least 0.005 mole glutamic acid or a salt thereof/kg dough or batter and/or
the presence of an isolated aspartate decarboxylase enzyme and aspartic acid or a salt thereof at a concentration of at least 0.005 mole aspartic acid or a salt thereof/kg dough or batter.

16. The dough or batter according to claim 15, comprising glutamic acid or a salt thereof at a concentration of at least 0.020 mole/kg dough or batter or comprising aspartic acid or a salt thereof at a concentration of at least 0.020 mole/kg dough or batter.
